SEQUENCE LISTING (10 sequences)

SEQ ID NO: 1 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - protein, 836 aa, Sus scrofa
MIPFLPVFSLFLLVVVNPANANGHYDKILAHSRIRGRDQGPNVCALQQILGTKKKYFSTCKNWYQGAICGKKTTVLYECCPGYMRMEGMKGCPAVLPIDHVYGTLGIVGATTTQRYSDVSKLREEIEGKGSFTYFAPSNEAWDNLDSDIRRGLESNVNVELLNALHSHMVNKRMLTKDLKNGMIIPSMYNNLGLFINHYPNGVVTVNCARIIHGNQIATNGVVHVIDRVLTQIGTSIQDFIEAEDDLSSFRAAAITSDILESLGRDGHFTLFAPTNEAFEKLPRGVLERIMGDKVASEALMKYHILNTLQCSEAITSGAVFETLEGNTIEIGCDGDSITVNGIKMVNKKDIVTNNGVIHLIDQVLIPDSAKQVIELAGNQQTTFTDLVAQLGLASALRPDGEYTLLAPVNNAFSDDTLSMDQRLLKLILQNHILKVKVGLNELYNGQKLETIGGKQLRVFVYRTAVCIENSCMVRGSKQGRNGAIHIFREIIKPAEKSLHEKLKQDKRFSIFLSLLEAADLKELLTQPGDWTLFVPTNDAFKGMTNEEKEILIRDKNALQNIILYHLTPGVFIGKGFEPGVTNILKTTQGSKIYLKGVNDTLLVNELKSKESDIMTTNGVIHVVDKLLYPADIPVGNDQLLEILNKLIKYIQIKFVRGSTFKEIPITVYTTKIITKVVEPKIKVIEGSLQPIIKTEGPMITKVKIEGEPELRLVKEGETVTEVIHGEPIIKKYTKIIDGVPVEITEKETREERIITGPEIKYTRISTSGGETEETLKKLLQEEVTKVTKFIEGGDGHLFEDEEIKRLLQGDTPVRKIQATKRVQGSRRRSREDRPQ

SEQ ID NO: 3 - protein, 836 aa, Canis familiaris
MIPFLPIFSLLLLVVVNPANANGHYDKILAHSRIRGRDQGPNVCALQQILGTKKKYFSTCRNWYQGAICGRKTTVLYECCPGYMRMEGMKGCPAVLPIDHVYGTLGIVGATTTQRYSDVSRLREEIEGKGSFTYFAPSNEAWDNLDSDIRRGLESNVNVELLNALHSHMVNNRMLTKDLKNGMIIPSMYNNLGLFINHYPNGVVTVNCARIIHGNQIATNGVVHVIDRVLTQIGTSIQDFIEAEDDLSSFRAAAITSDILESLGRDGHFTLFAPTNEAFEKLPRGVLERIMGDKVASEALMKYHLLNTLQCSEAIMGGAVFETLEGNTIEIGCDGDSITVNGIKMVNKKDIVTNNGVIHLIDQVLIPDSAKQVIELAGNQQTTFTDLVAQLGLASALRPDGEYTLLAPVNNAFSDDTLSMDQRLLKLILQNHILKVKVGLNELYNGQKLETIGGKQLRVFVYRTAVCIENSCMVRGSKQGRNGAIHIFREIIKPAEKSLHEQLKQDKRFSTFLSLLEAADLKELLTQPGDWTLFVPTNDAFKGMTNEEKEILIRDKNALQNIILYHLTPGVFIGKGFEPGVTNILKTTQGSKIYLKGVNDTLLVNEVKSKESDIMTTNGVIHVVDKLLYPADTPVGNDQLLEILNKLIKYIQIKFVRGSTFKEIPMTVYTTKIITKVVEPKIKVIEGSLQPIIKTEGPTITKVKIEGEPEFRLVKEGETVTEVIHGEPIIKKYTKIIDGVPVEITEKETREERIITGPEIKYTRISTGGGETEETLKKLLQEEVTKVTKFIEGGDGHLFEDEEIKRLLQGDTPVKKIQANKRVQGSRRRSREGRSQ

SEQ ID NO: 4 - protein, 779 aa, Bos taurus
MIPFLPVCSLFLLLVVNPANANGHYDKILAHSRIRGRDQGPNVCALQQILGTKKKYLSTCRNWYQGAICGKKTTVLYECCPGYMRMEGMKGCPAVMPIDHVYGTLGIVGATTTQSYSDVSKLREEIEGKGSFTYFAPSNEAWDNLDSDIRRGLESNVNVELLNALHSHMVNKRMLTKDLKNGMIIPSMYNNLGLFINHYPNGVVTVNCARIIHGNQIATNGVVHVIDRVLTQIGTSIQDFIEAEDELSSFRAAAITSDILETLGRDGHFTLFAPTNEAFEKLPRGVLERIMGDKVASEALMKYHILNTLQCSEAIMGGAVFETLEGNTIEIGCDGDSITVNGIKMVNKKDIVTNNGVIHLIDQVLVPDSAKQVIELAGNQQTTFTDLVAQLGLASALRPDGEYTLLAPVNNAFSDDTLSMDQRLLKLILQNHILKVKIGLNELYNGQKLETIGGKQLRVFVYRTAVCIENSCMVRGSKQGRNGAIHIFQEIIKPAEKSLHEKLKQDKRFSIFLSLLEAADLKELLTQPGDWTLFVPTNDAFKGMTNEEKEILIRDKNALQNIILYHLTPGVFIGKGFEPGVTNILKTTQGSKIYLKGVNDTLLVNELKSKESDIMTTNGVIHVVDKLLYPADTPVGNDQLLEILNKLIKYIQIKFVRGSTFKEIPITVYKPIIKKYTKIIDGVPVEITEKETREERIITGPEIKYTRISTTGGETEETLKKLLQEEVTKVTKFIEGGDGHLFEDEEIKRLLQGDTPVRKIQANKRVQASRRRSREDRPQ

SEQ ID NO: 5 - protein, 779 aa, Equus caballus
MIPFLPIFSLLLLFAVNPANANGHYDKILAHSRIRGRDQGPNVCALQQILGTKKKYFSTCRNWYQGAICGKKTTVLYECCPGYMRMEGMKGCPAVLPIDHVYGTLGIVGATTTQGYSDVSKLREEIEGKGSFTYFAPSNEAWDNLDPDIRRGLESNVNVELLNALHSHMVNKRMLTKDLKNGMIVPSMYNNLGLFINHYTNGVVTVNCARIIHGNQIATNGVVHVIDRVLTQIGTSIQDFIEAEDDLSSFRATAITSDILEALGRDGHFTLFAPTNEAFEKLPRGVLERIMGDKVASEALMKYHILNTLQCSEAIMGGAVFETLEGNTIEIGCDGDSITVNGIKMVNKKDIVTNNGVIHLIDQVLIPDSAKQVIELAGNQQTTFTDLVAQLGLASALRPDGEYTLLAPVNNAFSDDTLSMDQRLLKLILQNHILKVKVGLNELYNGQKLETIGGKQLRVFVYRTAVCIENSCMVRGSKQGRNGAIHIFREIIKPAEKSLHEKLKQDKRFSIFLSLLEAADLKELLTQPGDWTLFVPTNDAFKGMTNEEKEILIRDKNALQNIILYHLTPGVFIGKGFEPGVTNILKTTQGSKIYLKGVNDTLLVNELKSKESDIMTTNGVIHVVDKLLYPADTPVGNDQLLEILNKLIKYIQIKFVRGSSFKEIPMTVYKPIIKKYTKIIDGVPVEITEKETREERIITGPEIKYTRISTGGGETEETLKKLLQEEVTKVTKFIEGGDGHLLEDEEIKRLLQGDTPVRKMQANKRVQGSRRRSREGRSQ

SEQ ID NO: 6 - DNA, 3219 bp, Homo sapiens
agactctcaggttgatgcagtgttccctcccacaactctgacatgtatataaattctgagctctccaaagcccactgccagttctcttcggggactaactgcaacggagagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctataagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaacaggactttatacctcttttactgtaacaagtactcattaaaggaaattgaatgaaattaaaaaaaaaaa

SEQ ID NO: 7 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 8 - protein, 322 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 9 - protein, 348 aa, Mus musculus
MLASVAGPISLALVLLALCTRPAMGQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 10 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

(19) United States
(12) Patent Application Publication
(10) Pub. No.: US 2015/0273019 A1
(43) Pub. Date: Oct. 1, 2015

(54) METHOD OF TREATING WOUNDS

(71) Applicants: Douglas Hamilton, (US); Andrew Leask, (US)

(72) Inventors: Douglas Hamilton, London (CA); Christopher Elliott, Marie (CA); Jianjun Guan, Dublin, OH (US); Andrew Leask, London (CA)

(21) Appl. No.: 14/593,918

(22) Filed: Jan. 9, 2015

Related U.S. Application Data
(63) Continuation-in-part of application No. PCT/CA2013/000630, filed on Jul. 11, 2013.
(60) Provisional application No. 61/670,388, filed on Jul. 11, 2012.

(51) Int. Cl. A61K 38/18; A61L 27/24; A61K 38/17; A61L 27/26

(52) U.S. Cl. CPC A61K 38/18; A61L 27/26; A61L 27/24; A61K 38/177; A61L 2430/00; A61L 2300/414; A61L 2300/45

(57) ABSTRACT

A novel composition and method of treating a wound is provided. The method comprises administering a therapeutically effective amount of periostin and/or CCN2 proteins to the wound.

### PRIORITY CLAIMS AND RELATED PATENT APPLICATIONS

This application claims the benefit of priority, under 35 U.S.C. §120, from the US designation of International Application No. PCT/CA2013/000630, filed on Jul. 11, 2013, which claims benefit of priority from U.S. Provisional Application Ser. No. 61/670,388, filed on Jul. 11, 2012, the entire content of each of which is incorporated herein by reference in its entirety for all purposes.

### FIELD OF THE INVENTION

The present invention generally relates to wound healing, and in particular, relates to a combination of exogenous matricellular proteins to heal wounds such as skin or dermal wounds.

### BACKGROUND OF THE INVENTION

Non-healing or “chronic” dermal wounds are a significant clinical complication associated with aging, diabetes and immobility. Despite extensive research, reproducible clinical strategies for the closure of non-healing dermal wounds remain elusive, often resulting in limb amputation and peri-operative death. Although chronic wounds have different underlying etiologies (diabetes, venous insufficiency, deep tissue injury), all non-healing wounds are stalled in the inflammatory phase of wound repair and are unable to progress to the proliferative and remodeling phases required for healing. Identified as important mediators of normal wound healing, matricellular proteins interact with a discrete set of extracellular molecules, including growth factors and other ECM components, and contribute to specific phases of tissue repair.

Periostin has recently been classified as a matricellular protein. Unlike many other members of the matricellular protein family, periostin is normally expressed in adults, most commonly in collagen-rich tissues where its expression is often associated with fibroblasts. Periostin has been determined to be fibrogenic and prominently upregulated during extracellular matrix (ECM) remodeling, including following myocardial infarction, in bone marrow fibrosis and during pulmonary vascular remodeling. It therefore appears that periostin is an important regulator of fibroblast differentiation and ECM remodeling in both normal and pathological tissues.

Connective tissue growth factor (CCN2) is a matricellular protein that is not normally expressed in skin but is specifically induced in response to skin injury. CCN2 regulates ECM production and degradation, and stimulates angiogenesis. It promotes endothelial cell growth, migration, adhesion and survival and is thus implicated in endothelial cell function. It promotes myofibroblast differentiation in the presence of TGFβ. Although, CCN2 is considered to be a cofactor in fibrosis and not a fibrotic agent itself, it serves as a marker for severity of fibrosis in systemic sclerosis. CCN2 is required for maximal induction of α-SMA and collagen 1 by TGFβ. TGFβ-induced FAK and Akt activation is reduced in CCN2 null fibroblasts. Furthermore, CCN2 can also activate ERK through a syndecan-4-dependent mechanism.

Given the clinical need, it would be desirable to develop novel wound-healing therapies for chronic dermal wounds.

### SUMMARY OF THE INVENTION

It has now been found that the matricellular proteins, periostin and connective tissue growth factor (CCN2), provide novel and effective treatments of wounds, particularly chronic skin wounds.

Accordingly, in one aspect of the invention, a composition comprising a periostin protein, a CCN2 protein, or a combination thereof, is provided.

In another aspect of the invention, a method of treating a wound, such as a skin wound, is provided comprising administering a periostin protein, a CCN2 protein or a combination thereof, to the wound.

In a further aspect of the invention, an article of manufacture is provided comprising packaging and a composition comprising a periostin protein, a CCN2 protein or a combination thereof. The packaging is labeled to indicate that the composition is useful to treat a wound, such as a skin wound.

These and other aspects of the invention will become apparent in the following detailed description by reference to the following figures.

### BRIEF DESCRIPTION OF THE FIGURES

FIG. 1A illustrates the amino acid sequence of the human wildtype isoform of secreted periostin. FIG. 1B illustrates functionally equivalent isoform variants thereof and non-human functionally equivalent variants. FIG. 1C illustrates the mRNA sequence encoding human periostin.

FIG. 2A illustrates the amino acid sequence of isoforms of human CCN2. FIG. 2B illustrates a non-human CCN2 functionally equivalent variant thereof. FIG. 2C illustrates the mRNA sequence encoding human CCN2.

FIG. 3A illustrates mRNA levels of the matricellular proteins periostin (POSTN); FIG. 3B illustrates mRNA levels of CCN2; FIG. 3C illustrates mRNA levels of smooth muscle actin (ACTA2); FIG. 3D illustrates mRNA levels of collagen type I (COL1A2), in tissue isolated from areas at the edge and proximal to human chronic wounds and non-involved skin.

FIG. 4A graphically illustrates that dermal fibroblasts isolated from the edge of chronic wounds are phenotypically similar to healthy dermal fibroblasts (HDFa) and non-involved dermal fibroblasts with respect to proliferation. FIG. 4B graphically illustrates that dermal fibroblasts isolated from the edge of chronic wounds are phenotypically similar to healthy dermal fibroblasts (HDFa) and non-involved dermal fibroblasts with respect to collagen contraction.

FIG. 5 is a bar graph illustrating the results of an in vitro analysis of chronic wound fibroblast response to periostin and CCN2 matrices by measuring presence of smooth muscle actin.

FIG. 6A illustrates that periostin is not induced in chronic wounds. FIG. 6B illustrates that CCN2 is not induced in chronic wounds.

FIG. 7A illustrates the effects of periostin (PN)-, CCN2- and PN/CCN2-containing electrospun scaffolds on the treatment of excisional wounds in db/db diabetic mice over time. FIG. 7B illustrates a bar graph of wound area at day 7 following treatment.

FIG. 8 illustrates the influence of periostin, CCN2 and the combination of periostin and CCN2 on blood vessel density during wound healing in diabetic mice.

FIG. 9A graphically illustrates the effect of periostin, CCN2 and combination scaffolds on blood vessel density. FIG. 9B graphically illustrates the effect of periostin, CCN2 and combination scaffolds on blood vessel area.

### DETAILED DESCRIPTION OF THE INVENTION

A composition useful to treat wounds is herein provided comprising a periostin protein, a CCN2 protein or a combination thereof.

The term “wound” is used herein to refer to any injury to the skin which exhibits chronic wound behaviour, e.g. does not exhibit gene expression generally associated with wound healing such as upregulation of periostin, CCN2, smooth muscle actin and collagen type I. Exemplary wounds include, but are not limited to, a skin lesion such as an incision, laceration, abrasion, amputation, puncture wound, penetration wound and a chronic wound, including pressure, venous, and diabetic ulcers.

The term “treat” as it is used herein with respect to a wound refers to the amelioration or healing of a wound. Wound healing may be measured by the extent of wound closure, wherein at least about 20% wound closure over the initial wound size is indicative of wound healing, preferably at least about 30%, and more preferably at least about 40-50% or more wound closure over initial wound size.

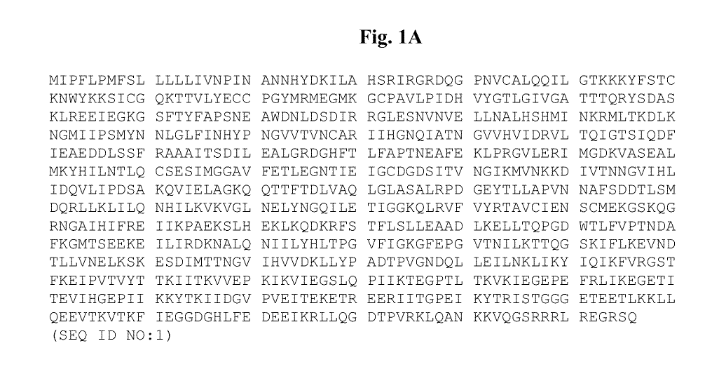

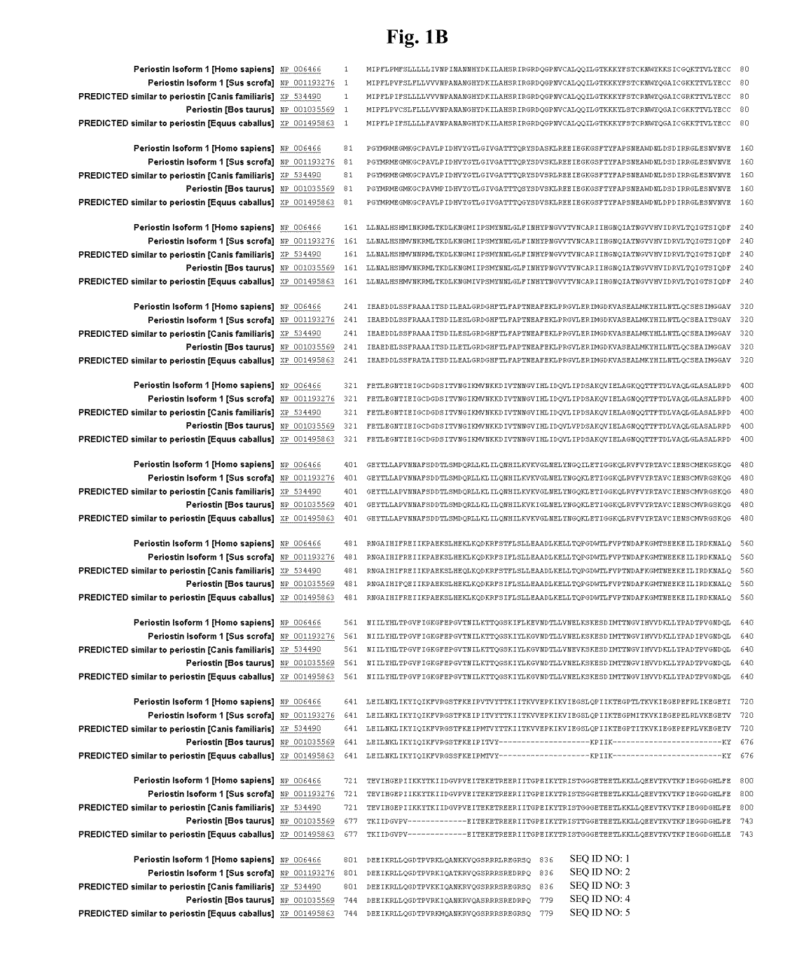

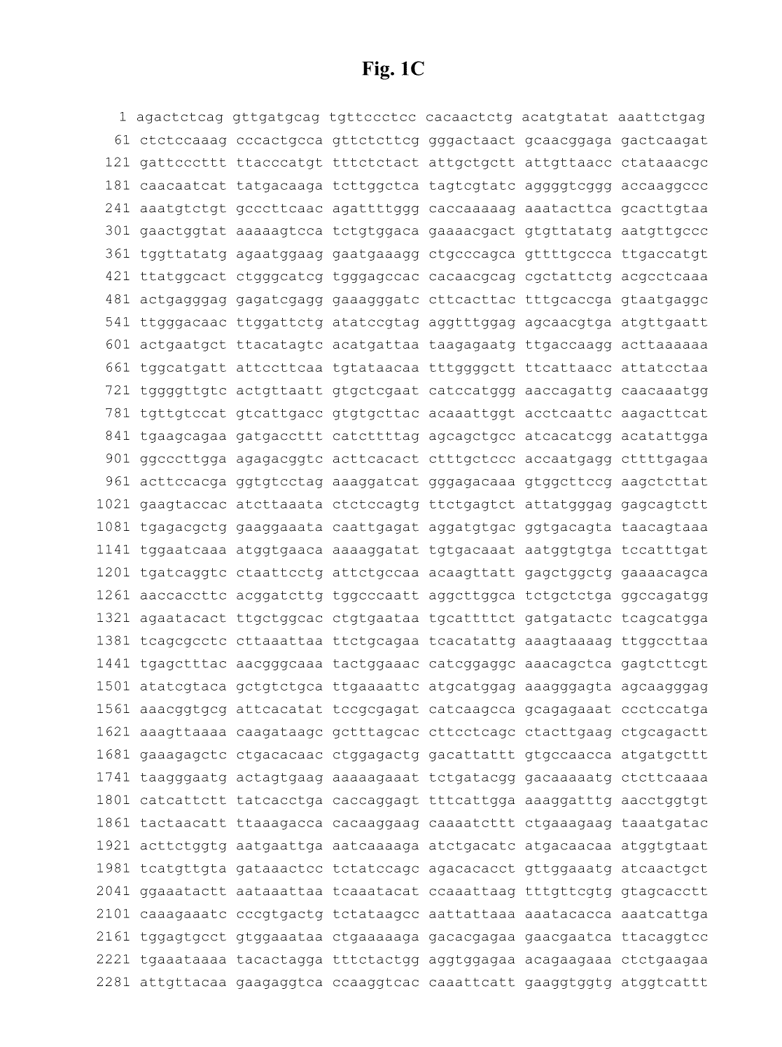

The term “periostin” also known as “osteoblast factor 2” or “OSF-2” is used herein to encompass mammalian and non-mammalian periostin proteins, including human and non-human periostin, and functionally equivalent forms thereof. Human periostin (e.g. the wildtype isoform) is an 836 amino acid protein as shown in FIG. 1A, and examples of functionally equivalent forms thereof include, for example, human isoforms 2, 3 and 4 and non-human forms as set out in FIG. 1B. FIG. 1C illustrates the sequence of human periostin mRNA.

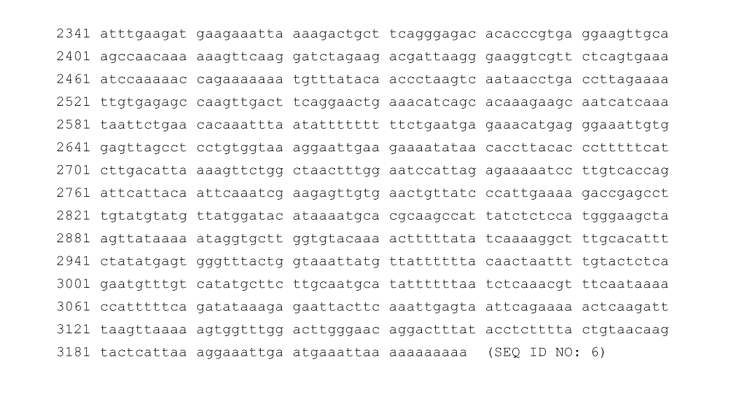

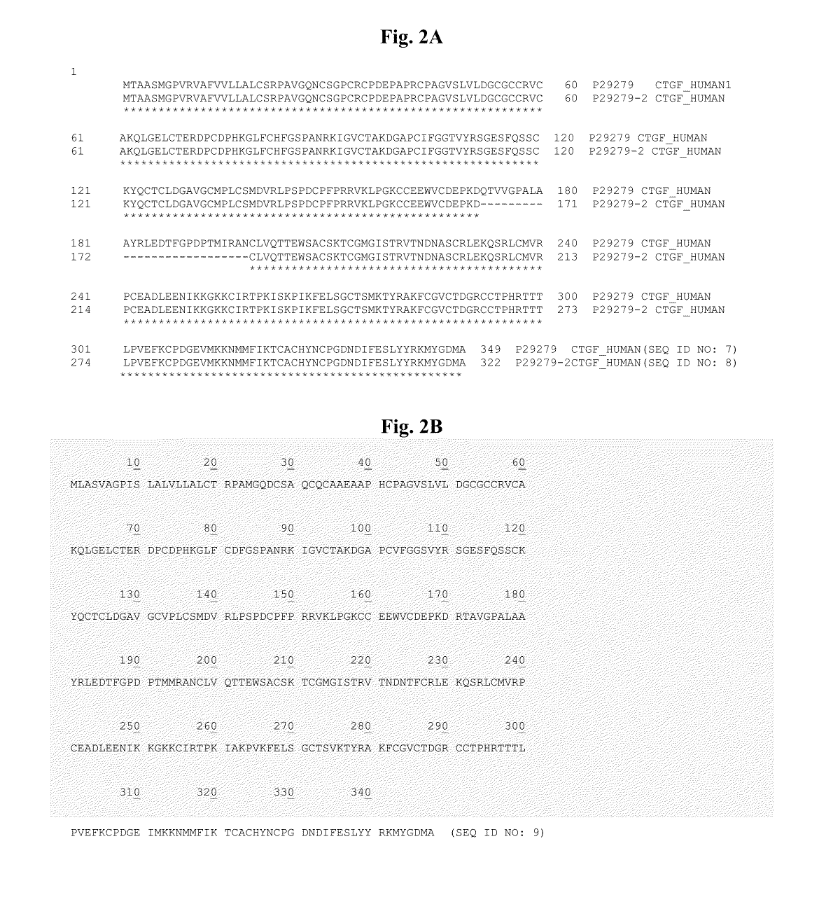

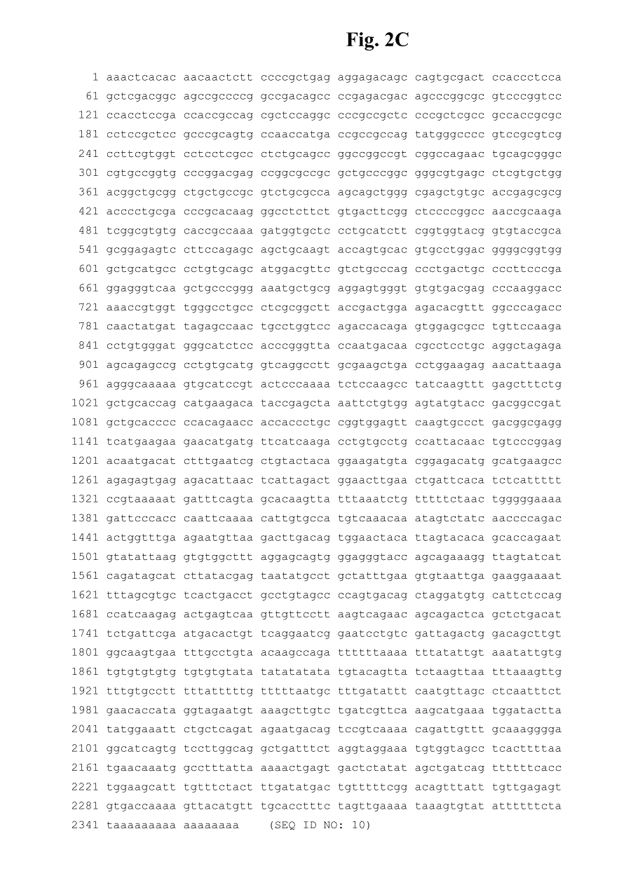

The term “connective tissue growth factor”, “CTGF” or “CCN2” is used herein to encompass mammalian and non-mammalian CCN2 proteins, including human and non-human CCN2, and functionally equivalent forms thereof. Human CCN2 (e.g. the wildtype isoform (isoform 1)) is a 349 amino acid protein as shown in FIG. 2A, and examples of functionally equivalent forms thereof include, for example, human isoform 2, also shown in FIG. 2A, and non-human forms such as those set out in FIG. 2B. FIG. 2C illustrates the sequence of human CCN2 mRNA.

The term “functional equivalent variants” as it relates to native periostin and CCN2, includes naturally or non-naturally occurring variants of the native protein that retain a level of wound-healing activity. The variant need not exhibit identical activity to the native protein, but will exhibit sufficient activity to render it useful for healing a wound, e.g. at least about 25% of the wound healing activity of the native protein, and preferably at least about 50% or greater of the wound healing activity of the native protein. Such functionally equivalent variants may result naturally from alternative splicing during transcription or from genetic coding differences and may retain significant sequence homology with the native wildtype protein, e.g. at least about 80% sequence homology, and preferably at least about 90% or greater sequence homology. Such variants can readily be identified using established cloning techniques employing primers derived from the native protein. Additionally, such modifications may result from non-naturally occurring synthetic alterations made to native protein to render functionally equivalent variants which may have more desirable characteristics for use in a therapeutic sense, for example, increased activity or stability. Non-naturally occurring variants of periostin and CCN2 include analogues, fragments and derivatives thereof. Periostin protein and CCN2 protein, thus, encompass native periostin and CCN2, as well as functionally equivalent variants thereof.

A functionally equivalent analogue of periostin or CCN2 in accordance with the present invention may incorporate one or more amino acid substitutions, additions or deletions. Amino acid additions or deletions include both terminal and internal additions or deletions to yield a functionally equivalent peptide. Examples of suitable amino acid additions or deletions include those incurred at positions within the protein that are not closely linked to activity, e.g. integrin binding or fibronectin/collagen/BMP-1 association in periostin, such as in the C-terminal region, pro-migratory activity of CTGF/CCN2 also located in the C-terminal domain, N-terminal domain containing motifs similar to that in insulin-like growth factor binding proteins (IGFBP) and TGF-β family member binding sites within Von-Willebrand factor (VWC) motifs. Amino acid substitutions within the protein, particularly conservative amino acid substitutions, may also generate functionally equivalent analogues thereof. Examples of conservative substitutions include the substitution of a non-polar (hydrophobic) residue such as alanine, isoleucine, valine, leucine or methionine with another non-polar (hydrophobic) residue; the substitution of a polar (hydrophilic) residue with another such as between arginine and lysine, between glutamine and asparagine, between glutamine and glutamic acid, between asparagine and aspartic acid, and between glycine and serine; the substitution of a basic residue such as lysine, arginine or histidine with another basic residue; or the substitution of an acidic residue, such as aspartic acid or glutamic acid with another acidic residue.

A functionally equivalent fragment in accordance with the present invention comprises a portion of the periostin or CCN2 protein which maintains a level of the function of the intact protein, e.g. with respect to wound healing, but not necessarily the same level of wound healing as the intact protein.

A functionally equivalent derivative of periostin or CCN2 in accordance with the present invention includes the native protein, or an analogue or fragment thereof, in which one or more of the amino acid residues therein is chemically derivatized. The amino acids may be derivatized at the amino or carboxy groups, or alternatively, at the side “R” groups thereof. Derivatization of amino acids within the peptide may render a peptide having more desirable characteristics such as increased stability or activity. Such derivatized molecules include for example, those molecules in which free amino groups have been derivatized to form, for example, amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids, for example: 4-hydroxyproline may be substituted for proline; 5-hydroxylysine may be substituted for lysine; 3-methylhistidine may be substituted for histidine; homoserine may be substituted for serine; and ornithine may be substituted for lysine. Terminal derivatization of the protein to protect against chemical or enzymatic degradation is also encompassed including acetylation at the N-terminus and amidation at the C-terminus of the peptide.

Periostin, CCN2 and functionally equivalent variants thereof, may be made using standard, well-established solid-phase peptide synthesis methods (SPPS). Two methods of solid phase peptide synthesis include the BOC and FMOC methods. Periostin and variants thereof may also be made using any one of a number of suitable techniques based on recombinant technology. It will be appreciated that such techniques are well-established by those skilled in the art, and involve the expression of periostin-encoding nucleic acid in a genetically engineered host cell. DNA encoding a periostin or CCN2 protein may be synthesized de novo by automated techniques well-known in the art given that the protein and nucleic acid sequences are known.

Once prepared and suitably purified, periostin, CCN2 or a functionally equivalent variant thereof, may be utilized in accordance with the invention for wound healing. In this regard, increasing the expression of periostin, CCN2 or variants at a target wound site, by administration of a periostin and/or a CCN2 protein, or by administration of oligonucleotides encoding one or both of a periostin and/or CCN2 protein, results in expression or over-expression of these proteins at a target wound site to promote wound healing. As one of skill in the art will appreciate, in a combination therapy, periostin and CCN2 may be administered to a wound together or separately.

The term “oligonucleotide” refers to an oligomer or polymer of nucleotide or nucleoside monomers consisting of naturally occurring bases, sugars, and intersugar (backbone) linkages. The term also includes modified or substituted oligonucleotides comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleiotides may contain at least one region of modified nucleotides that confer beneficial properties (e.g. increased nuclease resistance, increased uptake into cells), or two or more oligonucleotides of the invention may be joined to form a chimeric oligonucleotide. Other oligonucleotides of the invention may contain modified phosphorous, oxygen heteroatoms in the phosphate backbone, short chain alkyl or cycloalkyl intersugar linages or short chain heteroatomic or heterocyclic intersugar linkages. For example, oligonucleotides may contain phosphorothioates, phosphotriesters, methyl phosphonates, and phophorodithioates. Oligonucleotides of the invention may also comprise nucleotide analogs such as peptide nucleic acid (PNA) in which the deoxribose (or ribose) phosphate backbone in the DNA (or RNA), is replaced with a polyamide backbone similar to that found in peptides. Other oligonucleotide analogues may contain nucleotides containing polymer backbones, cyclic backbones, or acyclic backbones, e.g. morpholino backbone structures.

Such oligonucleotide molecules are readily synthesized using procedures known in the art based on the available sequence information. For example, oligonucleotides may be chemically synthesized using naturally occurring nucleotides or modified nucleotides as described above designed to increase the biological stability of the molecules or to increase the physical stability of the duplex formed with mRNA or the native gene, e.g. phosphorothioate derivatives and acridine substituted nucleotides. Selected oligonucleotides may also be produced biologically using recombinant technology in which an expression vector, e.g. plasmid, phagemid or attenuated virus, is introduced into cells in which the oligonucleotide is produced under the control of a regulatory region.

Once prepared, periostin- and/or CCN2-encoding oligonucleotides may be introduced into tissues or cells of a wound to promote wound healing using techniques well-established in the art which utilize vectors (retroviral vectors, adenoviral vectors and DNA virus vectors) or physical techniques such as microinjection. Therapeutic oligonucleotides may be directly administered in vivo or may be used to transfect cells in vitro which are then administered in vivo.

For wound treatment in accordance with an embodiment of the invention, periostin and/or CCN2 protein, or oligonucleotides encoding such proteins, may be administered alone or in combination with at least one pharmaceutically acceptable adjuvant. The expression “pharmaceutically acceptable” means acceptable for use in the pharmaceutical and veterinary arts, i.e. not being unacceptably toxic or otherwise unsuitable for administration to a mammal. Examples of pharmaceutically acceptable adjuvants include those used conventionally with peptide-based drugs, or with oligonucleotides, such as diluents, excipients and the like. Reference may be made to “Remington's: The Science and Practice of Pharmacy”, 21st Ed., Lippincott Williams & Wilkins, 2005, for guidance on drug formulations generally. As one of skill will appreciate, the selection of adjuvant may depend on the nature of the therapeutic compound, as well as the intended mode of administration of the composition. The compounds may, for example, be formulated for topical administration. Creams, lotions and ointments may be prepared for topical application using an appropriate base such as a triglyceride base. Such creams, lotions and ointments may also contain a surface active agent. The compounds may be combined with an adjuvant that provides structural support for topical administration. The compounds may also be formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Other adjuvants may also be included within the composition regardless of how it is to be administered, for example, preservatives, stabilizers, anti-oxidants, anti-microbial agents, colouring agents, and the like, to extend shelf-life of the composition.

In one embodiment of the invention, a periostin and/or CCN2 composition comprises a periostin and/or CCN2 protein combined with any adjuvant suitable for tissue engineering, e.g. an adjuvant comprising a meshwork that replicates physiological tissue, for example, skin, tendon, heart valves and other tissues. Adjuvants for this purpose include structural polymers. Examples of suitable structural polymers include, but are not limited to, naturally occurring structural polymers such as collagen, elastin, chitosan, tenascins and galectins, as well as synthetic polymers including but not limited to polyphosphazenes, poly(alpha-hydroxy esters) such as poly(glycolic acid) (PGA), poly(epsilon-caprolactone) (PCL), poly(L-lactic acid) (PLLA), poly(d,l-lactic acid) (PDLLA), and copolymers thereof, e.g. poly(DL-lactic-co-glycolic acid) (PLGA) such as PLGA5050 and PLGA8515, and hydrogels comprising alginate, agarose, fibrin, fibrinogen or cellulose. A composition comprising periostin- and/or CCN2-encoding oligonucleotides may also be combined with an adjuvant that is a biocompatible structural polymer as described for proteins.

A composition comprising a periostin and/or CCN2 protein, or oligonucleotides encoding these proteins, combined with a structural polymer adjuvant may be formed into a biocompatible scaffold prior to administration to a wound. Such a scaffold provides support for tissue growth on administration to a wound. For example, a biocompatible scaffold comprising periostin and/or CCN2 protein combined with a structural polymer may be prepared using a variety of well-established methods in the art, including for example, electrospinning. A scaffold may also be prepared directly into a wound, e.g. electrospun directly into the wound. A scaffold may be shaped and sized to fit any wound. Scaffolds may be prepared comprising both CCN2 and periostin with a suitable structural polymer, or comprising periostin or CCN2 alone. Scaffolds comprising periostin or CCN2 only may be layered within a wound to achieve the benefits of both periostin and CCN2 in the wound healing process, if desired. Scaffold thickness may be controlled during the process of making such scaffolds, and may be in the range of about 200 μm to 5 mm thick.

While not wishing to be limited to any particular mode of action, such scaffolds encourage cell recruitment, growth and differentiation during wound healing. A scaffold provides structural support and thereby enhances cell infiltration, while the matricellular proteins within the scaffold, i.e. periostin and/or CCN2, provide instructional cues for the cells to remake the tissue. Since periostin and CCN2 within the scaffold are ECM-associated cell-signaling proteins, they function to bind integrins, modify cell adhesion, and facilitate migration, differentiation, extracellular matrix synthesis, angiogenesis and vascular ingrowth to promote wound healing.

In another embodiment, the present composition may comprise both a periostin protein and a CCN2 protein which may advantageously provide greater wound healing efficacy over treatment with a periostin or CCN2 protein alone. In this regard, while not wishing to be limited to any particular mode of action, a combination of a periostin protein and a CCN2 protein may result in a synergistic effect on particular genes involved in wound healing as compared to the effect achieved by periostin or CCN2 alone, and may also impact genes to effect wound healing, either by upregulation or downregulation, that neither periostin nor CCN2 affect when used alone. For example, CCN2 activates pericyte progenitor cells, while periostin acts on dermal fibroblasts. The combination of CCN2 and periostin upregulates acting binding and cytoskeletal binding proteins, whereas CCN2 and periostin alone do not.

To treat a wound, a therapeutically effective amount of a periostin and/or a CCN2 protein is administered to a mammal at a target site. As used herein, the term “mammal” is meant to encompass, without limitation, humans, domestic animals such as dogs, cats, horses, cattle, swine, sheep, goats and the like, as well as non-domesticated animals. The term “therapeutically effective amount” is an amount of the periostin and/or CCN2 protein required to treat a wound by inducing a wound healing effect, while not exceeding an amount which may cause significant adverse effects. Dosages of periostin and/or CCN2 protein that are therapeutically effective will depend on many factors including, for example, the efficacy of the particular periostin and/or CCN2 proteins utilized for a given treatment, the nature of the wound to be treated as well as the particular individual being treated. Appropriate dosages of periostin and/or CCN2 protein for use are dosages sufficient to effect at least about 30% wound closure. In one embodiment, dosages within the range of about 10 ng/ml to 100 μg/ml of either of periostin or CCN2 are appropriate. If a periostin protein is combined with a CCN2 protein, dosages of each may be reduced from the dosage of either when used alone to achieve wound healing in view of the synergy of such a combination. In one example, a composition for use in wound healing may include 50 μg/ml of periostin and 50 μg/ml of CCN2. In a preferred embodiment, periostin and/or CCN2 may be combined with a structural polymer in a ratio of about 1:100000 to 1:500000 periostin and/or CCN2 to structural polymer by weight, e.g. about 1:225000 periostin and/or CCN2 to structural polymer by weight, may be utilized. If oligonucleotides encoding a periostin and/or CCN2 protein are used to treat a wound, it is expected that such oligonucleotides are administered to a wound in an amount that results in expression of at least about 10 ng/ml to 100 μg/ml protein.

As one of skill in the art will appreciate, the present compositions and methods may employ matricellular proteins in addition to periostin and/or CCN2 to achieve wound healing. Examples of such additional matricellular proteins include osteopontin, thrombospondins or galectins.

In another aspect of the present invention, an article of manufacture is provided. The article of manufacture comprises packaging material and a composition comprising a pharmaceutically acceptable adjuvant and a therapeutically effective amount of a periostin and/or CCN2 protein. The packaging material is labeled to indicate that the composition is useful to treat wounds.

The packaging material may be any suitable material generally used to package pharmaceutical agents including, for example, glass, plastic, foil and cardboard.

Embodiments of the invention are described by reference to the following examples which are not to be construed as limiting.

### Example 1

### Periostin and CCN2 are not Expressed in Chronic Wound Tissue

Materials and Methods:

Skin samples were obtained with informed consent from patients exhibiting non-healing skin wounds and undergoing elective lower extremity amputation for the affected limb. 18 patients were enrolled with a median age of 71.5 years (ranging from 34 to 88). Of these, four were female. The majority of patients were type-two diabetic (n=15) and one was type-one diabetic. Diagnosis of the patients' condition was almost exclusively peripheral vascular disease, making it likely that the samples collected were representative of arterial wounds. Sets of skin samples were collected from the wound site and proximal to the wound (within 500 μm), as well as from a non-involved region of the limb. At each site, samples were collected for histology, RNA isolation and cell culture, which were immersed in 10% neutral buffered formalin (Sigma Aldrich, St. Louis, Mo.), RNAlater® (Ambion, Carlsbad, Calif.) or growth media, respectively, until they could be further processed. For RNA isolation, tissues were snap frozen in liquid nitrogen and stored at −86° C. Snap frozen tissue samples were homogenized in 1 ml of TRIzol reagent (Invitrogen, Carlsbad, Calif.). Total RNA was extracted as per the manufacturer's recommendations. Real-time quantitative PCR was carried out on 50 ng of total RNA using TaqMan One-Step RT-PCR Master Mix and gene-specific TaqMan probes (Applied Biosystems, Carlsbad, Calif.). Postn (periostin), Collagen type I (COL1A2), CCN2, and smooth muscle actin (ACTA2) gene expression were normalized to the native control gene, 18S. PCR efficiency was verified to fall between 90 and 110% by dilution series, and relative expression was calculated using the DDCT method.

Results:

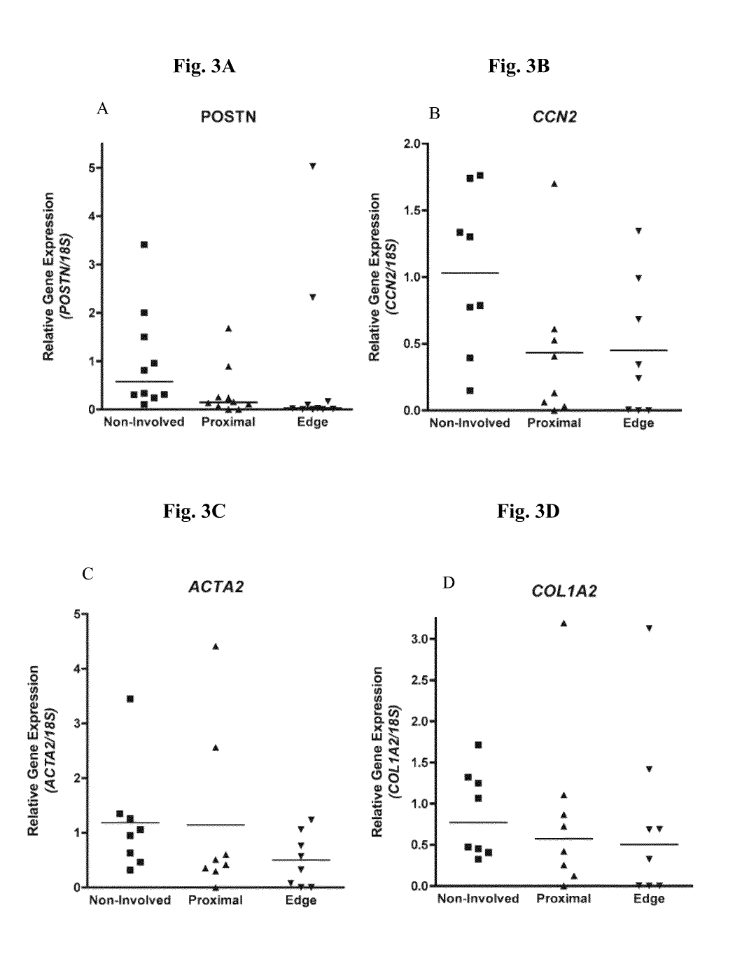

Although periostin and CCN2 are generally induced in wound healing (at edge of wound and the granulation tissue within the wound) to regulate downstream molecular events including smooth muscle actin (ACTA2) and collagen (COL1A2) expression during the healing process, neither periostin nor CCN2 mRNA were found to be induced in the present chronic wound tissue and this correlated with the absence of smooth muscle actin or collagen type 1 in the present chronic wound tissue (FIG. 3). Thus, it was concluded that the lack of periostin or CCN2 expression impaired the normal wound healing response to contribute to the wound becoming chronic.

### Example 2

### Smooth Muscle Actin (a-SMA) and Pericyte Activation (NG2) are not Expressed in Chronic Wound Tissue

Materials and Methods:

Tissues from chronic wound samples (as above) were stained as previously described (Jackson-Boeters et al. J Cell Commun Signal, 3(2):125-33, 2009). Sections were blocked with 10% horse serum and incubated with primary antibody overnight at 4° C. Smooth muscle actin and NG2 were detected on paraffin sections prepared from the tissues using the ABC kit (Vectorstain) following the manufacturer's instructions. The antibodies were diluted 1:3000 for rabbit anti-periostin (Kruzynska-Frejtag at al., 2004), and 1:500 for NG2. Signals were developed using DAB and hydrogen peroxide as the chromogen. Sections were counterstained with methyl green.

Results:

In normal healing, both pericytes (progenitor cells located on the outside of blood vessels) and dermal fibroblasts are recruited and differentiate into myofibroblasts in the granulation tissue. However, in the present chronic wound tissue, NG2 positive pericytes were not recruited from the blood vessels and no myofibroblast differentiation was observed in the granulation tissue. This correlates with no induction of periostin or CCN2 (see example 1). Thus, in human chronic wounds, the lack of expression of periostin and CCN2 also prevents normal wound healing by inhibiting pericyte recruitment and myofibroblast differentiation. In addition, no angiogenesis was evident in the wound bed, providing further evidence for a lack of pericyte recruitment.

### Example 3

### Dermal Fibroblasts Isolated from the Edge of Chronic Wounds are Phenotypically Similar to Healthy Dermal Fibroblasts (HDFa) and Non-Involved Dermal Fibroblasts

Materials and Methods:

Human dermal fibroblasts were isolated from non-involved and chronic wound edge tissue using an explant technique as previously described in Chen et al. 2008. Arthritis Rheum 58, 577-85. Cells were isolated from non-involved dermis and from the chronic wound edge and their proliferation rates compared to dermal fibroblasts from healthy human skin. Primary murine dermal fibroblasts were seeded at 2000 cells/well in 24 well plates in 10% FBS supplemented media. Media was changed every 48 hours throughout the course of the experiments. At the desired time-points media was completely aspirated and the plate was frozen at −80° C. Once all time-points were captured and all plates were frozen, the plates were allowed to thaw at room temperature. The CyQUANT cell proliferation assay kit (Invitrogen) was used to determine cell number as per the manufacturer's protocol. Briefly, 250 μL of working strength CyQUANT-GR dye was added to each well and incubated at room temperature for 5 minutes. From this, 200 μL of each sample was loaded into an opaque, clear bottom, 96 well assay plate. The dye was excited at 480 nm and emission at 520 nm was compared to a standard curve to obtain cell number.

In preparation for floating matrix gel contraction assays, 24 well plates were blocked with 1% BSA in PBS overnight at 4° C. Collagen was prepared as follows: 10% 0.2 M Hepes (pH 8), 40% collagen (Advanced BioMatrix) and 50% 2× Dulbecco's Modified Eagle Medium (High Glucose). Primary murine, or human, dermal fibroblasts were suspended in 0.5% FBS growth media and mixed 1:1 with the collagen preparation to get a final cell density of 100,000 cells/mL of collagen/media matrix. To each well of the blocked 24 well plate, 1 mL of collagen/cell mix was added and allowed to set at 37° C. Treatment with 5 ng/mL of recombinant transforming growth factor beta 1 (TGFβ1) and 1 ng/mL recombinant tumor necrosis factor alpha (TNFα) was carried out before the collagen gels had set. Once the collagen had set, wells were flooded with 1 mL 0.5% FBS growth media (with or without treatment). After 24 hours, gels were separated from the plate by gently running a P10 pipet tip around the wall of each well. Floating gels were allowed to contract for 24 hours. To determine the extent of gel contraction, gels were removed from the wells, blotted to remove excess media, and weighed.

Results:

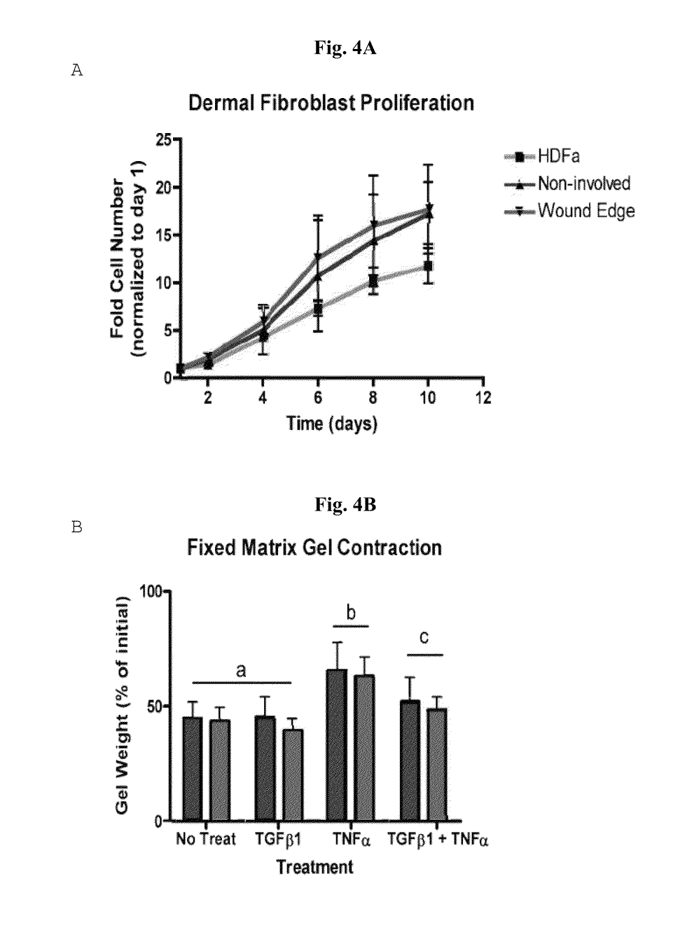

No significant differences in proliferation rates were evident between healthy human, non-involved or chronic wound edge fibroblasts, as shown in FIG. 4A demonstrating that in cell culture, chronic wound edge fibroblasts show normal proliferation. Similarly, wound edge fibroblasts were able to contract free floating collagen gels (FIG. 4B), a process that could be inhibited by the addition of tumour necrosis factor alpha, a cytokine over-expressed in chronic wounds. This demonstrates that chronic wound fibroblasts are able to exhibit the normal cell behaviours (proliferation and matrix contraction) necessary for normal wound healing. It was therefore concluded that the wound environment was inhibiting these cellular responses.

### Example 4

### Analysis of Chronic Wound Fibroblast Response to Periostin and CCN2 Matrices

Materials and Methods:

Human dermal fibroblasts were isolated from non-involved and chronic wound edge tissue using an explant technique as above described. Excised tissue were immediately transferred to Dulbecco's modified Eagle's medium (High Glucose) supplemented with 10% fetal bovine serum and 2% AA (200 U penicillin, 200 mg streptomycin, 0.5 mg/ml amphotericin B) (Gibco, Carlsbad, Calif.) Skin was washed with five changes of medium then incubated at 37° C., 5% CO2 to allow fibroblasts to migrate onto the culture surface Skin was removed and cells were cultured for two to three passages before use. Periostin, CCN2 and periostin+CCN2 were coated onto collagen on tissue culture plastic or 7% polyacrylamide gels. The latter mimics the stiffness of granulation tissue. Matrix-coated flexible polyacrylamide substrates were created on glass coverslips using methods described previously (Bhana et al. Biotechnol Bioeng. 2010. Vo. 105(6):1148-60). Polyacrylamide gels were prepared at 7%, which correlates to a stiffness of 4800 Pa.

Discussion of Results:

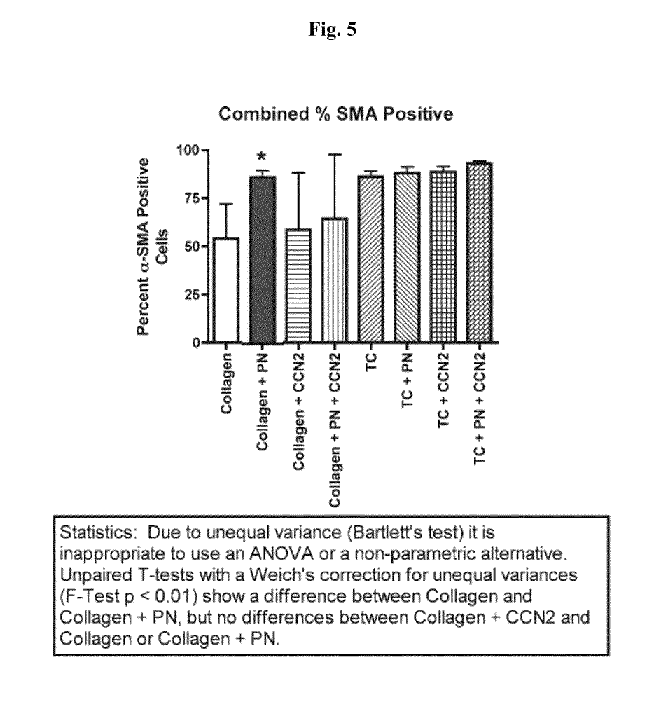

Results are shown in FIG. 5. When cultured on stiff substrates (TC), all chronic wound fibroblasts differentiated into myofibroblasts (as indicated by presence of smooth muscle actin) irrespective of whether periostin, CCN2 or combinations of periostin+CCN2 were present. Myofibroblast differentiation on stiff substrata is a well-described phenomenon. On low stiffness substrates, periostin was the most potent stimulator of myofibroblast differentiation indicating that the combination of periostin+CCN2 must induce other effects in tissue not evident in these assays.

### Example 5

### Periostin- and CCN2-Containing Electrospun Scaffolds Enhance Wound Closure

Materials and Methods:

Breeding colonies of db/db and wild-type (WT) mice have been established in a C57/BL6 background and represent a murine model of type II diabetes. The B6.BKS(D)-Leprdb/J mouse was identified initially in 1966 as an obese mouse that develops hyperglycemia with blood glucose values over 20 mM at 10 wk of age. db/db mice develop diabetes due to a deficiency in leptin receptor activity as the mice are homozygous for a point mutation in the leptin gene as described in Lerman et al., Am J Pathol 2003, 162, (1), 303-12. Using a standard blood sugar monitor, glucose was measured in blood samples extracted through the tail vein. On average, the db/db mice colony exhibited glucose levels of 25 mM. Excisional wounds in db/db mice exhibit a statistically significant delay in wound closure and decreased granulation-tissue formation. The db/db model has been used to validate treatments for chronic wounds. db/db (10 weeks of age, with glucose levels above 20 mM) and WT sex- and age matched mice (glucose levels under 10 mM) were anesthetized with an intraperitoneal injection containing ketamine (100 mg/kg) and xylazine (5 mg/kg). It should be noted that typically at 10 weeks of age, WT mice weigh approximately 25 g, but db/db mice weigh around 45 g by comparison. The backs of the mice were cleaned, shaved, depilated with Nair® and sterilized with betadine solution. 6 mm full-thickness skin wounds were made along the dorsorostral back skin in each animal. All procedures were approved by the University Council on Animal Care at the University of Western Ontario.

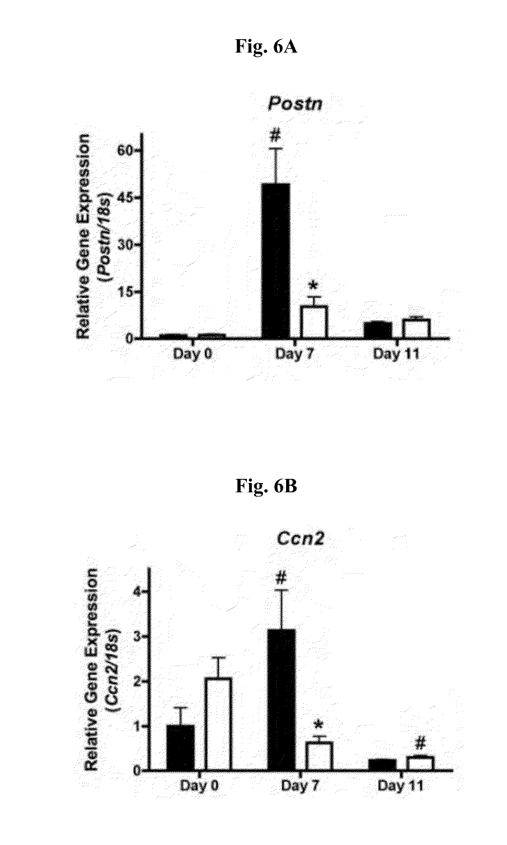

Total RNA was extracted from wild-type and db/db full thickness excisional punch wounds at 7 and 11 days post wounding. Excised tissue was used for day 0 samples. Quantitative RT-PCR was carried out with probes specific for Postn and CCN2. Periostin and CCN2 were not induced during healing of the full-thickness skin wounds in diabetic db/db mice as shown in FIG. 6. Data was normalized to the native control gene, 18s. Compared to wild-type mice, db/db mice show a failure to appropriately induce fibrotic genes during wound repair (#=p<0.05 compared to day 0, *=p<0.05 compared to wild-type, n=5).

For scaffolds, Type I collagen (Sigma-Aldrich) was dissolved in 1,1,1,3,3,3-hexafluoro-2-propanol to make a 15% (w/v) solution. Periostin (R&D Systems) was dissolved in PBS to a 1 mg/ml solution. For periostin-collagen scaffolds, 20 μl periostin solution was mixed with 20 μL BSA solution (100 mg/mL in PBS) and 3 ml of collagen solution. For CCN2-collagen scaffolds, 20 μl CCN2 solution (0.5 mg/mL dissolved in PBS) was mixed with 20 μL BSA solution and 3 ml of collagen solution. For periostin/CCN2-collagen scaffolds, 10 μl periostin and 10 μl CCN2 was mixed with 20 μL BSA solution and 3 ml of collagen solution. Each mixture was injected at a speed of 1 ml/h by a syringe pump into a capillary charged with a voltage of +15 kV. The generated nanofibres were collected on a negatively charged (−10 kV) rotation mandrel. Control scaffolds contained 20 μl of BSA in PBS. To crosslink the scaffolds, they were immersed in 5% glutaraldehyde/ethanol solution for 30 min. The scaffolds were spun onto aluminum foil, and a 6-mm biopsy punch was used to cut the scaffolds to the same diameter as the wound in the skin of the mice.

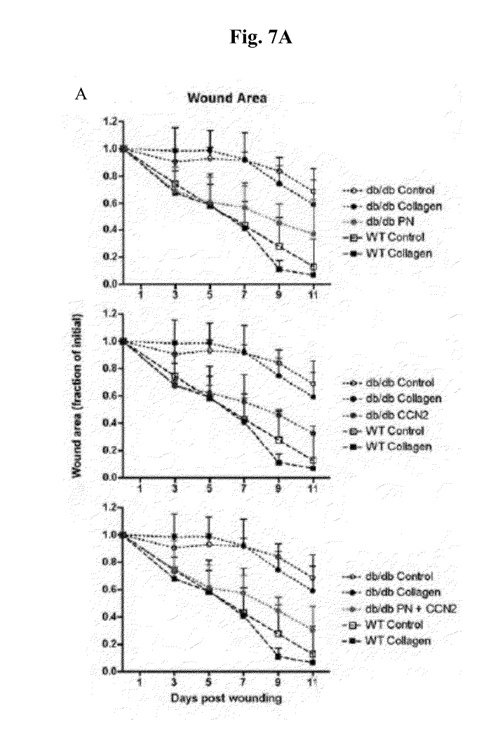

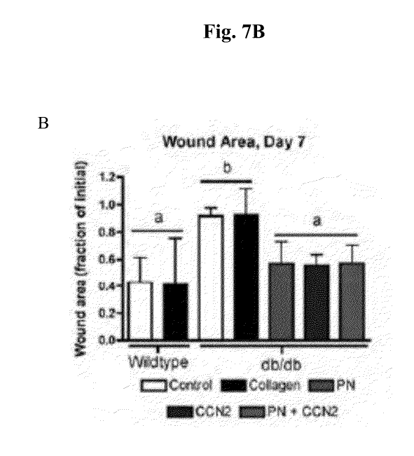

Each scaffold was rinsed 3 times with 100% ethanol and vacuum-dried overnight. Two 6-mm diameter scaffolds, one on top of the other, were placed in each wound. Wounds were either treated with a periostin-collagen scaffold, a CCN2-collagen scaffold, a periostin- and CCN2-collagen scaffold, a collagen/BSA scaffold or no scaffold. The scaffolds were held in place by coagulation of the blood resulting from creation of the wounds. By using diabetic and wild-type mice, it could be assessed whether or not the scaffolds increased the speed of wound repair (wild-types) as well as whether they can rescue the dermal phenotype of the db/db mice. Wounds were photographed daily until 11 days post-wounding. At least 5 wounds per treatment were followed for the 11-day course. Wound area was assessed from photographs using Northern Eclipse v7.0 software (Empix Imaging Inc., Mississauga, Ontario) and expressed as a fraction of initial area. Mice were caged individually following wounding and were sacrificed at various time points for histological analysis. Quantification of wound area from photographs shows the recovery of wound closure rate in the db/db wounds treated with PN or CCN2 scaffolds (FIG. 7A). At day 7, it is clear that collagen alone is not sufficient to explain the increase in closure rate (FIG. 7B). PN, CCN2 and PN+CCN2 treated wounds exhibited similar closure rates, which were statistically equivalent to wild-type closure rates.

Discussion of Results:

Addition of periostin, CCN2, or combinations of periostin+CCN2, in scaffolds, significantly increased the rate of wound closure compared to collagen alone or control empty wounds as shown in FIG. 7. Thus, the addition of periostin and/or CCN2 has a positive effect on closure of chronic diabetic wounds.

### Example 6

### Delivery of Periostin and CCN2 in Combination Enhances Wound Contraction

Materials and Methods:

Full thickness excisional wounds in db/db diabetic mice as described in Example 5 were photographed at day 16 following treatments as described for analysis of wound contraction.

Discussion of Results:

In the presence of periostin+CCN2, increased levels of wound contraction were evident in comparison to either periostin or CCN2 alone. This demonstrates that when both CCN2 and periostin were delivered locally to the wound bed, additional effects were present enhancing wound contraction, that neither periostin nor CCN2 alone were able to promote. It therefore appears that the proteins together exhibit enhanced wound treatment.

### Example 7

### Periostin and CCN2 in Combination Prologs Expression of Smooth Muscle Actin

Materials and Methods:

Smooth muscle actin, CCN2 and periostin were detected on paraffin sections of healing tissue from full thickness excisional wounds in db/db diabetic mice using the ABC kit (Vectorstain) following the manufacturer's instructions. The antibodies were diluted 1:3000 for rabbit anti-periostin (Kruzynska-Frejtag at al., 2004), and 1:500 for mouse anti-Ki67 (DB). Signals were revealed by using DAB, and then hydrogen peroxide as the chromogen. Sections were counterstained with methyl green. Trichrome staining was carried out using established techniques.

Discussion of Results:

In the presence of CCN2+periostin, increased levels of smooth muscle actin expression were evident compared to treatment with either periostin or CCN2 alone. This data indicates that when both CCN2 and periostin were delivered locally to the wound bed, additional effects were present enhancing wound contraction, that neither periostin nor CCN2 alone were able to promote. In addition, in the presence of CCN2, periostin levels were higher indicating that CCN2 stimulates host cells to secrete periostin.

### Example 8

### Periostin+CCN2 Increases the Average Density of Blood Vessels

Materials and Methods:

Smooth muscle actin was detected on paraffin sections of dermal tissue from full thickness excisional wounds in db/db diabetic mice using the ABC kit (Vectorstain) following the manufacturer's instructions. The antibodies were diluted 1:3000 for rabbit anti-periostin (Kruzynska-Frejtag at al., 2004), and 1:500 for mouse anti-Ki67 (DB). Signals were revealed by using DAB, and then hydrogen peroxide as the chromogen. Sections were counterstained with methyl green.

Discussion of Results:

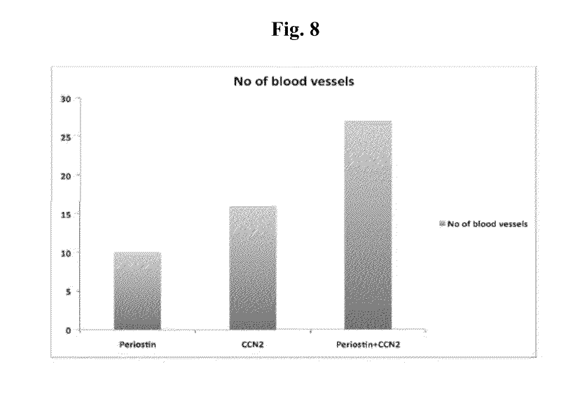

The number of blood vessels in the granulation tissue from wounds to which periostin, CCN2 or periostin+CCN2 were delivered via scaffolds was examined. Addition of periostin scaffolds stimulated the lowest number of blood vessels. CCN2 alone stimulated more, which is consistent with its known role as an angiogenic factor, but the combination of periostin+CCN2 stimulated the most angiogenesis as shown in FIG. 8. This clearly demonstrates that periostin and CCN2 when delivered together enhance cellular processes leading to increased blood vessel development in the healing tissue.

### Example 9

### Differential Gene Expression from Periostin and CCN2 Wound Treatment

Materials and Methods:

Total RNA from the collagen, periostin, CCN2 and periostin/CCN2 treated wounds of 3 db/db mice were used to generate cDNA as follows. RNA quality was assessed using the Agilent 2100 Bioanalyzer (Agilent Technologies Inc., Palo Alto, Calif.) and the RNA 6000 Nano kit (Caliper Life Sciences, Mountain View, Calif.). Single stranded complimentary DNA (sscDNA) was prepared from 200 ng of total RNA as per the Ambion WT Expression Kit for Affymetrix GeneChip Whole Transcript WT Expression Arrays (Applied Biosystems) and the Affymetrix GeneChip WT Terminal Labeling kit and Hybridization (Affymetrix, Santa Clara, Calif.). Total RNA was first converted to cDNA, followed by in vitro transcription to make cRNA. 5.5 μg of single stranded cDNA was synthesized, end labeled and hybridized, for 16 hours at 45° C., to Mouse Gene 2.0 ST arrays. A GeneChip Fluidics Station 450 performed all liquid handling steps and GeneChips were scanned with the GeneChip Scanner 3000 7G (Affymetrix) using Command Console v3.2.4. Probe level (.CEL file) data were summarized to gene level data in Partek Genomics Suite v6.6 (Partek, St. Louis, Mo.) using the RMA algorithm. Partek was used to determine gene level ANOVA p-values and fold changes. Gene Ontology (GO), KEGG Pathway and SwissProt and Protein Information Resource (SP PIR) keyword enrichments were generated using DAVID Bioinformatics Resources 6.7, NIAID/NIH. Enrichments were filtered by a p-value and the Benjamini-Hochberg method was used to control for false discovery rate (FDR). Differentially expressed genes were selected based on an ANOVA p-value of less than 0.05 and 1.5 fold increase or decrease from db/db Collagen samples.

Gene cluster analysis at day 7 post implantation of periostin/collagen, CCN2/collagen or periostin/CCN2/collagen scaffolds in db/db full thickness excisional wounds was analyzed. Each scaffold type activates specific gene expression patterns that are unique indicating that they function through different mechanisms despite their similar effects on wound closure rate. CCN2-containing scaffolds caused an upregulation of a number of genes associated with intracellular signaling (G-protein coupled receptor and cell surface receptor, as shown in Table 1) and down-regulation of a number of genes involved with fatty acid synthesis, obesity and PPARγ signaling. PPARγ signaling directly opposes the profibrotic actions of TGFβ. Periostin scaffolds promote profibrotic gene expression, while suppressing the actions of PPARγ. Periostin scaffolds had a strong influence on genes associated with early inflammation, the defense response (see Table 1), and down-regulation of several genes associated with protease inhibitors (Table 2). This indicates that periostin influences wound closure through modulation of the inflammatory phase of healing.

When a combination of periostin/CCN2/collagen was administered to a wound, a pattern of differential gene expression was observed which included a large number of genes that were not changed in either of the individual periostin or CCN2 treatments alone. The combination of periostin and CCN2 stimulated the highest number of gene changes in relation to several biological processes required for wound healing as shown in Table 1 and Table 2 below, exemplifying the fact that the combination of periostin and CCN2 acts synergistically to provide a more potent treatment than either of periostin or CCN2 when used alone. For example, contractile fiber genes were upregulated (Table 1) by both periostin/collagen and CCN2/collagen scaffolds, individually, by a certain degree (e.g. 3 genes, and 5 genes, respectively), while treatment with a combination periostin/CCN2/collagen scaffold affected the expression of 29 contractile fiber genes, an effect that is clearly synergistic, e.g. greater than the additive effect of treatment with periostin and treatment with CCN2. Contractile gene expression is essential for proper wound healing. Robust enrichments of contractile gene sets was further shown [Table 2: (Muscle proteins: PN—3 genes; CCN2—7 genes; Periostin-CCN2—17 genes). In addition, the combination scaffold resulted in downregulation of genes associated with re-epithelialization (keratinization, epidermal cell differentiation, etc, table 1], which indicates that in combination, CCN2 and periostin increase the rate of epithelial closure, which neither of periostin nor CCN2 alone does.

TABLE 1

| Gene cluster | Periostin | | CCN2 | | Periostin + CCN2 | |
|---|---|---|---|---|---|---|
| designation | Up | Down | Up | Down | Up | Down |
| Acute phase | 3 | | 0 | | 0 | |
| response | | | | | | |
| Acute | 3 | | 0 | | 0 | |
| inflammatory | | | | | | |
| response | | | | | | |
| Inflammatory | 3 | | 0 | | 0 | |
| response | | | | | | |
| Defense response | 5 | | 0 | | 0 | |
| Keratinization | | 0 | | 0 | | 7 |
| Keratinocyte | | 0 | | 0 | | 12 |
| differentiation | | | | | | |
| Epidermal cell | | 0 | | 0 | | 12 |
| differentiation | | | | | | |
| Epidermis | | 0 | | 0 | | 20 |
| development | | | | | | |
| Actin binding | 0 | | 0 | | 19 | |
| Cytoskeletal | 0 | | 0 | | 22 | |
| binding proteins | | | | | | |
| Contractile fiber | 3 | | 5 | | 29 | |
| Myosin complex | 0 | | 3 | | 10 | |
| Ion binding | 0 | | 0 | | 38 | |
| Metal ion binding | 0 | | 0 | | 37 | |
| G-protein | 0 | 0 | 31 | 3 | 0 | 0 |
| coupled receptor | | | | | | |
| protein signaling | | | | | | |
| pathway | | | | | | |
| Cell surface | 0 | | 32 | | 0 | |
| receptor linked | | | | | | |
| signal | | | | | | |
| transduction | | | | | | |
| Extracellular | 12 | | 0 | | 0 | |
| compartment | | | | | | |
| Oxidation | | 6 | | 20 | | 30 |
| reduction | | | | | | |
| Biological | | 0 | | 0 | | 20 |
| adhesion | | | | | | |

TABLE 2

| | Term | | | | | | | |
|---|---|---|---|---|---|---|---|---|
| | PN | | | | | CCN2 | | |
| KEYWORDS | Count | P value | Genes | Fold Enrich | Benjamini | Count | P value | Genes |

Gene upregulations

| heart | | | | | | 2 | 0.03327873 | ACTC1,TNN [symbol] |
|---|---|---|---|---|---|---|---|---|
| cardiac muscle | | | | | | 2 | 0.03327873 | ACTC1, TNN [symbol] |
| thick filament | 2 | 0.02448359 | MYH3, MYH [symbol] | 78.4791209 | 0.23865699 | 2 | 0.04771681 | MYH3, MYH7 |
| skeletal muscle | | | | | | | | |
| muscle protein | 3 | 0.00407211 | ACTC1, MYH [symbol] | 30.6068571 | 0.051018 | 7 | 3.10E−08 | ACTC1, TNN [symbol] |
| sarcoplasmic reticulum | | | | | | | | |
| sodium/potassium transport | | | | | | | | |
| myogenesis | | | | | | | | |
| myosin | 2 | 0.08579899 | MYH3, MYH8 | 21.7069909 | 0.49878845 | 3 | 0.01345175 | MYL65, MYH |
| EF hand | | | | | | | | |
| LIM domain | | | | | | | | |
| actin-binding | | | | | | | | |
| kelch repeat | | | | | | | | |
| calcium binding | | | | | | | | |
| calmodulin-binding | Gene cluster changes induced in vivo depending | | | | | | | |
| | on the constituents proteins of the scaffolds. | | | | | | | |
| motor protein | | | | | | | | |
| methylation | 3 | 0.06606856 | ACTC1, MYH [symbol] | 6.92462831 | 0.44277795 | | | |
| immunoglobulin domain | | | | | | | | |
| cytoskeleton | | | | | | | | |
| ion transport | | | | | | | | |
| coiled coil | | | | | | | | |
| sensory transduction | | | | | | 4 | 0.04386538 | VMN1R135, [symbol] |
| extracellular matrix | | | | | | 4 | 0.04596299 | FMOO, KERA |
| leucine-rich repeat | | | | | | 5 | 0.01978552 | FMOO, KERA |
| g-protein coupled receptor | | | | | | 25 | 1.02E−10 | VMN2R43, V [symbol] |
| transducer | | | | | | 25 | 2.28E−10 | VMN2R43, V [symbol] |
| receptor | | | | | | 30 | 3.68E−09 | VMN2R43, V [symbol] |
| disulfide bond | 13 | 0.00135859 | MUC2, SKINT | 2.68589944 | 0.02583108 | 17 | 0.0184664 | FMOO, KERA |
| transmembrane | | | | | | 30 | 0.01036404 | VMN2R43, V [symbol] |
| acute phase | 3 | 9.46E−04 | REG3B, SAA1 | 63.7642857 | 0.02400781 | | | |
| Lectin | 4 | 0.00388755 | REG3B, ZG16 | 12.2183062 | 0.05822127 | | | |
| Secreted | 11 | 2.19E−04 | REG3B, MUC | 3.95158954 | 0.00837983 | | | |
| signal | 16 | 1.54E−04 | MUC2, SKINT | 2.74809043 | 0.01182223 | | | |
| glycoprotein | 13 | 0.02897664 | MUC2, SKINT | 1.84207937 | 0.24649586 | | | |

Gene down regulation

| | | | | | | | | |
|---|---|---|---|---|---|---|---|---|
| keratinization | | | | | | | | |
| gap junction | | | | | | | | |
| tandem repeat | | | | | | | | |
| Fatty acid biosynthesis | | | | | | 5 | 4.45E−04 | SCD1, ELOVL [symbol] |
| Serine protease inhibitor | 4 | 0.00147737 | SERPINA3M, | 17.4185366 | 0.08623921 | 6 | 4.49E−04 | SERPINA3M, |
| lipid synthesis | | | | | | 10 | 7.74E−08 | SCD1, FAR2, [symbol] |
| intermediate filament | | | | | | | | |
| protease inhibitor | 6 | 1.19E−05 | SERPINA3M, | 19.6557798 | 0.00145159 | 6 | 0.00162634 | SERPINA3M, |
| electron transfer | | | | | | | | |
| keratin | | | | | | 5 | 0.01042624 | KRTAP12-1, [symbol] |
| lipid degradation | | | | | | 7 | 3.48E−05 | LPL, ENPP2, [symbol] |
| lipid metabolism | | | | | | 12 | 9.08E−09 | FAR2, SLC27 [symbol] |
| antibiotic | | | | | | | | |
| microsome | | | | | | 4 | 0.04324481 | ACSL1, FA2H, |
| Serine protease | 3 | 0.07451259 | TMPRSS11E, | 6.53195122 | 0.57633904 | | | |
| Antimicrobial | | | | | | | | |
| multifunctional enzyme | | | | | | 4 | 0.01042883 | PCX, HSD3B6 |
| nadp | 3 | 0.05794535 | FM03, CYP2I | 7.54394366 | 0.59759769 | 7 | 8.56E−04 | FAR2, FASN, |
| Acyltransferase | | | | | | 5 | 0.03301431 | AWAT1, ALA [symbol] |
| heme | 3 | 0.05794535 | HPX, HBB-B1 | 7.54394366 | 0.59759769 | 7 | 8.56E−04 | HBA-A2, CYP |
| cell junction | | | | | | | | |
| Monooxygenase | | | | | | | | |
| nad | | | | | | 7 | 0.00209644 | GPD1, HSD3 [symbol] |
| lyase | | | | | | 4 | 0.06935636 | CYP17A1, FA [symbol] |
| oxidoreductase | 6 | 0.01983577 | ALDH1A3, F [symbol] | 3.74559441 | 0.4572312 | 20 | 9.95E−08 | SCD1, GPD1, |
| cell adhesion | | | | | | | | |
| iron | 4 | 0.05787174 | HPX, HBB-B1 | 4.44959502 | 0.64618617 | 10 | 9.32E−04 | SCD1, HBA-A |
| cleavage on pair of | | | | | | | | |
| basic residues | | | | | | | | |
| calcium | | | | | | | | |
| endoplasmic reticulum | | | | | | 16 | 2.63E−04 | SCD1, H5D3B |
| Signal-anchor | | | | | | | | |
| Secreted | 11 | 0.00459395 | MUP7,SPINK | 2.76611268 | 0.17076433 | 24 | 6.02E−04 | RETNLA, ENP |
| Protease | | | | | | | | |
| signal | 14 | 0.0534162 | MUP7, SPINK | 1.68320539 | 0.67248259 | 38 | 0.00647985 | RETNLA, FGF |
| disulfide bond | 12 | 0.06783232 | MUP7, SPINK | 1.73550425 | 0.57554926 | 31 | 0.0080133 | RETNLA, FGF |
| cell membrane | | | | | | 20 | 0.07280948 | SLC27A1, SL [symbol] |
| hydrolase | | | | | | 19 | 0.04322296 | LPL, ENPP2, [symbol] |
| membrane | | | | | | 64 | 2.22E−04 | SLC16A14, SL [symbol] |
| glycoprotein | | | | | | 40 | 0.01561195 | FGFR2, SKINT |
| transmembrane | | | | | | 57 | 0.00360509 | SLC27A1, SL [symbol] |
| obesity | | | | | | 3 | 8.84E−04 | LEP, RETN, E [symbol] |
| diabetes mellitus | | | | | | 2 | 0.03832966 | LEP, RETN |
| oxygen carrier | | | | | | 2 | 0.03832966 | HBA-A2, HBB |
| blood | | | | | | 2 | 0.04581872 | HBA-A2, HBB |
| lipid droplet | 2 | 0.0271151 | PUN1, PLIN4 | 71.416 | 0.48867188 | 3 | 0.00259942 | PLIN1, PLIN4, |
| erythrocyte | | | | | | 2 | 0.06062356 | HBA-A2, HBB |
| oxygen transport | | | | | | 2 | 0.08240426 | HBA-A2, HBB |
| vldl | | | | | | 2 | 0.08240426 | LPL, APOC1 |
| chromoprotein | | | | | | 4 | 0.00369599 | HBA-A2, CYP |
| metalloprotein | | | | | | 5 | 9.44E−04 | HBA-A2, CYP |
| pyridoxal phosphate | | | | | | 3 | 0.07952245 | ALAS2, FASN, |
| chloride | | | | | | 3 | 0.09838633 | CLIC3, ACE2, |
| peroxisome | | | | | | 4 | 0.03906659 | FAR2, ACSL1, |
| manganese | | | | | | 6 | 0.00553112 | PCX, B3GALT |
| Symport | | | | | | 4 | 0.05342465 | SLC1A5, SLC1 |
| polymorphism | | | | | | 5 | 0.06714719 | HBA-A2, B3G |
| transmembrane protein | | | | | | 9 | 0.0084758 | FGFR2, SLC1 [symbol] |
| mitochondrion | | | | | | 11 | 0.089068 | PCX, ACSL1, [symbol] |
| transport | | | | | | 19 | 0.06238397 | SLC27A1, SL [symbol] |
| thiol protease inhibitor | 2 | 0.05870366 | 2010005H15 | 32.4618182 | 0.55960097 | | | |

| | Term | | | | | | |
|---|---|---|---|---|---|---|---|
| | CCN2 | | PN + CCN2 | | | | |
| KEYWORDS | Fold Enrich | Benjamini | Count | P value | Genes | Fold Enrich | Benjamini |

Gene upregulations

| heart | 58.3464052 | 0.32368654 | 4 | 3.51E−05 | ACTC1, TNNC | 57.9205191 | 0.00102378 |
|---|---|---|---|---|---|---|---|
| cardiac muscle | 53.34S4052 | 0.32368654 | 4 | 3.51E−05 | ACTC1, TNNC | 57.9205191 | 0.00102378 |
| thick filament | 40.3936652 | 8.34540446 | 5 | 2.18E−06 | MYH1, MYH3 | 50.1235261 | 1.27E−04 |
| skeletal muscle | | | 5 | 2.18E−06 | ACTC1, NRAP | 50.1235261 | 1.27E−04 |
| muscle protein | 36.7582353 | 8.06E−07 | 17 | 2.02E−22 | OBSCN, ACT [symbol] | 44.3091971 | 3.54E−20 |
| sarcoplasmic reticulum | | | 5 | 1.14E−05 | SLN, SRL, JPH | 34.2950442 | 5.00E−04 |
| sodium/potassium transport | | | 2 | 0.0593499 | ATP1B1, ATP | 32.580292 | 0.43080789 |
| myogenesis | | | 3 | 0.01100197 | MYF6, MURC | 18.6173097 | 0.12915104 |
| myosin | 16.7590738 | 0.2092284 | 6 | 2.83E−05 | MYH1, MYL3 | 16.6367448 | 9.89E−04 |
| EF hand | | | 3 | 0.01912899 | TNNC2, TNH [symbol] | 13.9629823 | 0.20175016 |
| LIM domain | | | 6 | 2.36E−04 | NRAP, XIRP2, | 10.7113289 | 0.00588837 |
| actin-binding | | | 17 | 1.74E−11 | MYH1, MYH3 | 9.80291971 | 1.52E−09 |
| kelch repeat | | | 5 | 0.001799 | KBTBD5, KLH | 9.58243881 | 0.02823978 |
| calcium binding | | | 3 | 0.04983119 | TNNC2, TNN [symbol] | 8.31837242 | 0.39162105 |
| calmodulin-binding | Gene cluster changes induced in vivo depending | | 6 | 0.00114764 | MYH1, MYH3 | 7.59152434 | 0.02208062 |
| | on the constituents proteins of the scaffolds. | | | | | | |
| motor protein | | | 6 | 0.00251027 | MYH1, MYL3 | 6.35713014 | 0.03326856 |
| methylation | | | 9 | 2.94E−04 | ACTC1, RND1 | 5.30719688 | 0.00641611 |
| immunoglobulin domain | | | 11 | 0.00210856 | OBSCN, IGFN | 3.23596579 | 0.0303133 |
| cytoskeleton | | | 13 | 0.00168307 | ACTC1, LDB3 | 2.90596086 | 0.02904831 |
| ion transport | | | 10 | 0.02323017 | SLC38A3, CA [symbol] | 2.40002151 | 0.22669182 |
| coiled coil | | | 21 | 0.03907607 | MYH1, MYH3 | 1.58010654 | 0.33656466 |
| sensory transduction | 5.02504925 | 0.37281152 | | | | | |
| extracellular matrix | 4.93068213 | 0.3590879 | | | | | |
| leucine-rich repeat | 4.77379679 | 0.22878679 | | | | | |
| g-protein coupled receptor | 4.651999 | 1.06E−08 | | | | | |
| transducer | 4.47441758 | 1.19E−08 | | | | | |
| receptor | 3.19544207 | 1.27E−07 | | | | | |
| disulfide bond | 1.80781693 | 0.241887 | | | | | |
| transmembrane | 1.50406047 | 0.19482605 | | | | | |
| acute phase | | | | | | | |
| Lectin | | | | | | | |
| Secreted | | | | | | | |
| signal | | | | | | | |
| glycoprotein | | | | | | | |

Gene down regulation

| | | | | | | | |
|---|---|---|---|---|---|---|---|
| keratinization | | | 6 | 2.92E−05 | SPRR2I-PS, H | 15.912656 | 5.93E−04 |
| gap junction | | | 4 | 0.00678678 | GJB4, PANX3 | 10.1472009 | 0.06681038 |
| tandem repeat | | | 3 | 0.04108245 | MTAP2, RPTI | 9.2125903 | 0.26481616 |
| Fatty acid biosynthesis | 13.8618012 | 0.01346669 | 7 | 1.25E−04 | ELOVL4, ELO [symbol] | 8.8788008 | 0.00205578 |
| Serine protease inhibitor | 9.33135889 | 0.01188549 | 11 | 1.32E−06 | WFDC12, SE [symbol] | 7.8269568 | 6.96E−05 |
| lipid synthesis | 12.8816739 | 8.25E−06 | 13 | 1.22E−07 | FA2H, LSS, 4 [symbol] | 7.66164917 | 1.08E−05 |
| intermediate filament | | | 9 | 3.75E−05 | KRT80, KRT5, | 7.09618442 | 7.07E−04 |
| protease inhibitor | 7.01992136 | 0.02445963 | 13 | 3.56E−07 | SPINK12, SE [symbol] | 6.95874558 | 2.35E−05 |
| electron transfer | | | 3 | 0.07719505 | CYP3A13, CY | 6.48293391 | 0.41152849 |
| keratin | 5.36983557 | 0.09250112 | 10 | 1.05E−04 | KRTAP12-1, [symbol] | 5.35288121 | 0.001852 |
| lipid degradation | 11.3 | 0.00185337 | 7 | 0.00231371 | LIPK, LIPM, P [symbol] | 5.16993464 | 0.0274139 |
| lipid metabolism | 11.252521 | 1.93E−06 | 12 | 2.21E−05 | FAR2, AWAT [symbol] | 5.14821223 | 5.31E−04 |
| antibiotic | | | 5 | 0.01623105 | DEFB6, WFD [symbol] | 5.11810572 | 0.12628824 |
| microsome | 5.10114286 | 0.26938956 | 8 | 0.00163649 | AADAC, CYP [symbol] | 4.66771242 | 0.02037934 |
| Serine protease | | | 13 | 2.58E−05 | KLK6, KLK8, [symbol] | 4.62501993 | 5.67E−04 |
| Antimicrobial | | | 5 | 0.02896455 | DEFB6, WFD [symbol] | 4.29017686 | 0.20405294 |
| multifunctional enzyme | 8.79507389 | 0.08884468 | 4 | 0.07672206 | PCX, HSD3B6 | 4.02389002 | 0.41745777 |
| nadp | 6.28661972 | 0.01808109 | 9 | 0.00308062 | FAR2, FASN, | 3.6980116 | 0.03336969 |
| Acyltransferase | 4.11382488 | 0.24044971 | 9 | 0.00522844 | AWAT1, SPT [symbol] | 3.38785579 | 0.05385287 |
| heme | 6.28661972 | 0.01808109 | 8 | 0.01112905 | CYP3A13, HP | 3.28712142 | 0.09379044 |
| cell junction | | | 22 | 4.20E−06 | CLDN4, FER [symbol] | 3.27454315 | 1.38E−04 |
| Monooxygenase | | | 6 | 0.03784322 | CYP3A13, CY | 3.24146696 | 0.25247486 |
| nad | 5.28224852 | 0.02936101 | 9 | 0.00862479 | HSD3B6, ALD | 3.10720501 | 0.08120944 |
| lyase | 4.18126464 | 0.34890452 | 6 | 0.0583079 | CAR12, FASN | 2.86949534 | 0.34122713 |
| oxidoreductase | 4.45904096 | 7.06E−06 | 28 | 1.99E−06 | HSD3B6, ALC | 2.85611774 | 8.75E−05 |
| cell adhesion | | | 16 | 0.00236798 | MPZL2, PTPR | 2.45669075 | 0.02684562 |
| iron | 3.97285269 | 0.0164108 | 13 | 0.00944671 | ALOXE3, CYP | 2.36293853 | 0.0827786 |
| cleavage on pair of | | | 9 | 0.05203269 | DSG1B, PRR [symbol] | 2.20637667 | 0.31700505 |
| basic residues | | | | | | | |
| calcium | | | 27 | 3.58E−04 | GALNT3, HR [symbol] | 2.15506558 | 0.00554294 |
| endoplasmic reticulum | 3.00952381 | 0.00929238 | 25 | 6.35E−04 | HSD3B6, SGP | 2.15141612 | 0.00879046 |
| Signal-anchor | | | 14 | 0.02252169 | GALNT3, AB [symbol] | 2.01691277 | 0.16659319 |
| Secreted | 2.15541247 | 0.01415533 | 49 | 3.94E−06 | RETNLA, KER | 2.01336187 | 1.48E−04 |
| Protease | | | 17 | 0.01448831 | KLK6, CPA4, [symbol] | 1.94870116 | 0.1168735 |
| signal | 1.54580087 | 0.06689258 | 88 | 1.17E−07 | RETNLA, LYP [symbol] | 1.72878238 | 1.55E−05 |
| disulfide bond | 1.60120928 | 0.07836419 | 72 | 5.05E−06 | RETNLA, MP [symbol] | 1.70147476 | 1.48E−04 |
| cell membrane | 1.48895005 | 0.35285765 | 47 | 0.00135448 | SLC16A14, L [symbol] | 1.60086459 | 0.01773217 |
| hydrolase | 1.6153619 | 0.27713329 | 39 | 0.0092338 | ATP10B, ABH | 1.51700654 | 0.08374994 |
| membrane | 1.48208254 | 0.00940569 | 143 | 4.83E−09 | 9530008L14 [symbol] | 1.51507825 | 1.28E−06 |
| glycoprotein | 1.41698413 | 0.12546507 | 93 | 1.88E−05 | MRZL2, LYPD | 1.50728214 | 4.97E−04 |
| transmembrane | 1.38803295 | 0.0442427 | 117 | 6.14E−04 | 9530008L14 [symbol] | 1.30351908 | 0.00896686 |
| obesity | 63.7642857 | 0.01698635 | | | | | |
| diabetes mellitus | 51.0114286 | 0.26532417 | | | | | |
| oxygen carrier | 51.0114286 | 0.26532417 | | | | | |
| blood | 42.5095238 | 0.27548976 | | | | | |
| lipid droplet | 38.2585714 | 0.0340564 | | | | | |
| erythrocyte | 31.8821429 | 0.33212932 | | | | | |
| oxygen transport | 23.187013 | 0.37479979 | | | | | |
| vldl | 23.187013 | 0.37479979 | | | | | |
| chromoprotein | 12.7528571 | 0.04287082 | | | | | |
| metalloprotein | 11.3864796 | 0.0153584 | | | | | |
| pyridoxal phosphate | 6.37642857 | 0.37153006 | | | | | |
| chloride | 5.6262605 | 0.41611683 | | | | | |
| peroxisome | 5.31369048 | 0.26150864 | | | | | |
| manganese | 5.27704433 | 0.06028518 | | | | | |
| Symport | 4.67994758 | 0.30612221 | | | | | |
| polymorphism | 3.25327988 | 0.34492459 | | | | | |
| transmembrane protein | 3.10204633 | 0.07910648 | | | | | |
| mitochondrion | 1.77571429 | 0.391497 | | | | | |
| transport | 1.54235701 | 0.3320484 | | | | | |
| thiol protease inhibitor | | | | | | | |

Evidence that periostin/CCN2 is a more potent scaffold for inducing gene changes than either periostin or CCN2 on their own. [symbol]
indicates data missing or illegible when filed

### Example 10

### Effect of Periostin, CCN2 and Combination Treatments on Blood Vessels

Methods:

6 mm punch excisional skin wounds were made in wild-type and db/db sex-matched adult mice (12 weeks of age) as described in Example 5. For assessment of angiogenesis and vascular ingrowth, some wounds were left untreated (control) while others received: 2× collagen type I scaffolds, 2× periostin/collagen type I, 2×CCN2/collagen type I or 1× periostin/collagen type I and 1×CCN2/collagen type I containing scaffolds as described in Example 5. 8 mm diameter scaffolds were inserted into each wound immediately following wounding. Animals were sacrificed at day 11, tissue retrieved, embedded and sections labelled with antibodies specific to CD146. Images were captured and blood vessel density and blood vessel area calculated using ImageJ software. Statistical analysis was performed using a one-way ANOVA with Bonferroni adjustment for multiple comparisons.

Results:

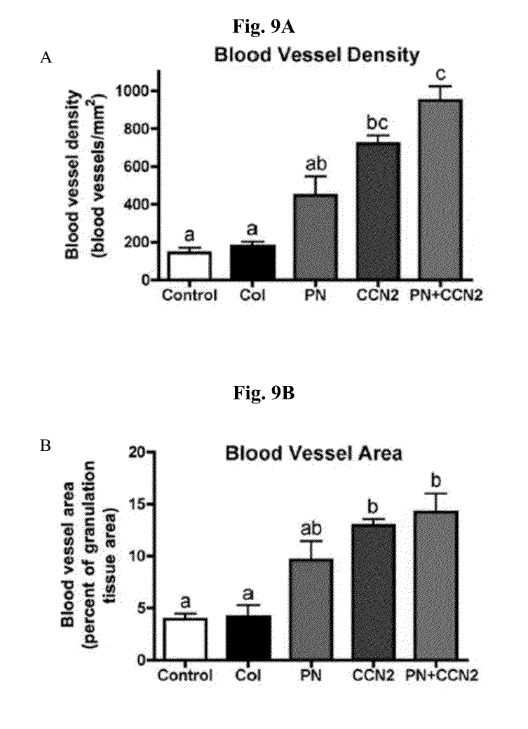

Blood vessel density was statistically similar between control, collagen-treated and periostin-treated wounds. However, blood vessel density within the granulation tissue of wounds treated with both periostin/collagen type I and CCN2/collagen type I electrospun scaffolds was significantly increased (n=3, p<0.01, one-way ANOVA) as shown in FIG. 9A. The total area occupied by blood vessels was also significantly higher in wounds receiving the combination periostin/collagen type I and CCN2/collagen type I (n=3, p<0.01, one-way ANOVA) (FIG. 9B).

1. A composition comprising a combination of a periostin protein and a CCN2 protein.

2. The composition of claim 1, wherein the periostin protein is selected from the group consisting of periostin and a functionally equivalent variant thereof, and the CCN2 protein is selected from the group consisting of CCN2 and a functionally equivalent variant thereof.

3. The composition of claim 2, wherein the periostin functionally equivalent variant exhibits at least about 80% sequence homology to periostin, and the CCN2 functionally equivalent variant exhibits at least about 80% sequence homology to CCN2.

4. The composition of claim 1, comprising a pharmaceutically acceptable adjuvant.

5. The composition of claim 1, wherein the composition is suitable for topical administration.

6. The composition of claim 4, comprising an adjuvant which is a structural polymer.

7. The composition of claim 6, wherein the structural polymer is a naturally occurring structural polymer selected from the group consisting of collagen, elastin, chitosan, tenascins and galectins; or a synthetic polymer selected from the group consisting of polyphosphazenes, poly(glycolic acid) (PGA), poly(epsilon-caprolactone) (PCL), poly(L-lactic acid) (PLLA), poly(d,l-lactic acid) (PDLLA), copolymers thereof, and hydrogels comprising fibrin, fibrinogen, alginate, agarose or cellulose.

8. The composition of claim 6, comprising a ratio of about 1:100000 to 1:500000 periostin and CCN2 protein to structural polymer by weight.

9. The composition of claim 6, which is a biocompatible scaffold.

10. A method of treating a wound, comprising administering to the wound a composition comprising a combination of a periostin protein and a CCN2 protein.

11. The method of claim 10, wherein the periostin protein is selected from the group consisting of periostin and a functionally equivalent variant thereof, and the CCN2 protein is selected from the group consisting of CCN2 and a functionally equivalent variant thereof.

12. The method of claim 11, wherein the periostin functionally equivalent variant exhibits at least about 80% sequence homology to periostin, and the CCN2 functionally equivalent variant exhibits at least about 80% sequence homology to CCN2.

13. The method of claim 12, wherein the wound is a chronic skin wound.

14. The method of claim 12, wherein the composition comprises a pharmaceutically acceptable adjuvant.

15. The method of claim 14, wherein the adjuvant is a structural polymer.

16. The method of claim 15, wherein the structural polymer is a naturally occurring structural polymer selected from the group consisting of collagen, elastin, chitosan, tenascins and galectins; a synthetic polymer selected from the group consisting of polyphosphazenes, poly(glycolic acid) (PGA), poly(epsilon-caprolactone) (PCL), poly(L-lactic acid) (PLLA), poly(d,l-lactic acid) (PDLLA), copolymers thereof, and hydrogels comprising fibrin, fibrinogen, alginate, agarose or cellulose; or a combination thereof.

17. The method of claim 15, wherein the composition comprises a ratio of about 1:100000 to 1:500000 periostin and CCN2 protein to structural polymer by weight.

18. The method of claim 10, wherein the composition comprises about 10 ng/ml to 100 μg/ml of periostin and CCN2 proteins.

19. A method of treating a wound, comprising administering to the wound a CCN2 protein.
